(19) United States
(12) Patent Application Publication
(10) Pub. No.: US 2010/0022909 A1
(43) Pub. Date: Jan. 28, 2010

(54) METHODS AND DEVICES FOR MEASURING CORE BODY TEMPERATURE

(75) Inventor: Alexander V. Padiy, Geldrop (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/531,312

(22) PCT Filed: Feb. 15, 2008
(86) PCT No.: PCT/IB2008/050566

§ 371 Date: Sep. 15, 2009

Related U.S. Application Data
(60) Provisional application No. 60/894,915, filed on Mar. 15, 2007.

(51) Int. Cl. A61B 5/01

(52) U.S. Cl. 600/549

(57) ABSTRACT

A core body temperature measurement device includes temperature sensor (12, 12f, 12b), a head-mountable mechanical frame or pad (42, 62, 64, 72, 82, 102, 112) configured to operatively couple the temperature sensor with skin overlaying an arterial blood rich superficial region (STA, PAA) disposed near to an auricle and outside of an ear canal or other skin (CAR) overlaying the carotid artery or a major arterial branch thereof, and a readout controller (10, 48, 68, 90) configured to acquire a temperature measurement using the temperature sensor and to output a core body temperature based on the acquired temperature measurement.

The following relates to the medical arts. It finds particular application in measuring core body temperature, and is described with particular reference thereto. However, the following finds more general application in measuring core body temperature-related values suitable for use in medical diagnostic, treatment monitoring, and related medical applications.

Core body temperature is an important medical vital sign. Unlike other vital signs such as heart rate or blood pressure, core body temperature is relatively insensitive to variations due to psychological or emotional state. Thus, core body temperature can be a good indicator of a medical problem. Moreover, a shift in core body temperature of only a few degrees Celsius away from the typical range can be life-threatening in and of itself, providing further motivation for monitoring this critical vital sign.

Unfortunately, core body temperature has heretofore been more difficult to measure than other vital signs such as heart rate or blood pressure. The core body temperature is defined as the temperature of blood flowing through the heart. However, for clinical purposes the core body temperature is typically taken as the brain temperature, since this value is typically close to the cardiac core temperature, and elevated brain temperature is a clinically serious condition that would be useful to monitor in clinical settings. As used herein, core body temperature is taken to correspond to the brain temperature. A rectal thermometer is also sometimes used to measure core body temperature, under the assumption that the rectal temperature is a suitable surrogate for the core body temperature. However, rectal temperature may differ substantially from core body temperature of the heart or brain. Insertion of a rectal thermometer is also uncomfortable for the patient, and rectal thermometry is not well-suited for extended monitoring over a period of hours, days, or longer.

To precisely measure core body temperature, a temperature sensor can be inserted into brain vasculature using a suitable catheter instrument. Although precise, this approach is clinically problematic because it is invasive and can produce disadvantageous side effects such as infection, vascular clotting, or so forth.

Core body temperature can also be estimated by measuring forehead temperature. This is the basis for the home diagnostic of placing a hand over the forehead of the patient to determine whether a fever is present. As a measure of core body temperature, this technique is inexact at best. A more precise core body temperature estimate can be obtained by placing a thermocouple, thermistor, or other temperature sensor into contact with the forehead. However, the temperature acquired by such sensors can differ substantially from the core body temperature due to temperature drop across the skin and other intervening tissue. This temperature drop is not constant, but varies significantly as a function of sweat, room temperature, skin thickness, and other factors.

Core body temperature is also sometimes estimated as the reading of an oral thermometer. However, the oral temperature also provided by an oral thermometer can vary substantially depending upon where the thermometer tip or other temperature sensor is placed within the patient's mouth. Respiration can also affect the measured temperature. More fundamentally, the oral temperature can differ substantially from the core body temperature due to the substantial distance and large amount of intervening tissue between the orally-placed temperature sensor and the brain.

Thermometers are also known which are inserted into the ear canal to contact the tympanic membrane, also known colloquially as the ear drum. The tympanic membrane has relatively close proximity to the brain and thus reflects the core body temperature relatively accurately. However, the shape of the ear canal varies from person to person, and in some instances access to the tympanic membrane may be impeded or blocked by curvature of the ear canal. Another potential source of error is wax buildup in the ear canal. Physical contact with the tympanic membrane by the thermometer can also promote ear infection, which can be a serious medical condition. Core body temperature measurement via the tympanic membrane is also not well suited for extended monitoring over a period of hours, days, or longer.

Abreu, U.S. Published Application 2004/0059212, discloses a recently developed technique for measuring core body temperature that overcomes some of these difficulties. The approach of Abreu is based on identification of a thermally conductive pathway to the brain, called a “brain tunnel” in US 2004/0059212, located between the eyes proximate to an orbit or eye socket. By using contact thermometry at the location of this “brain tunnel,” a relatively accurate core body temperature reading can be non-invasively obtained.

Unfortunately, the identified brain tunnel has a small external cross-section near the eye orbit, which makes the accuracy of the core body temperature measurement strongly dependent upon accurate placement of the temperature sensor. Deviations of as little as one or two millimeters can adversely affect the core body temperature measurement. Additionally, placement of a temperature sensor near the eye can be discomforting for the patient, can lead to eye infection, and is not well-suited for extended monitoring over a period of hours, days, or longer. The eye-based temperature sensor can also interfere with other diagnostic operations involving access of the patient's eye, nose, or other nearby facial regions.

The following provides a new and improved apparatuses and methods which overcome the above-referenced problems and others.

In accordance with one aspect, a core body temperature measurement device includes a temperature sensor, a head-mountable mechanical frame or pad configured to operatively couple the temperature sensor with skin overlaying an arterial blood rich superficial region disposed near to an auricle and outside of an ear canal, and a readout controller configured to acquire a temperature measurement using the temperature sensor and to output a core body temperature based on the acquired temperature measurement.

In accordance with another aspect, a core body temperature measurement method includes operatively coupling a temperature sensor with skin overlaying an arterial blood rich superficial region disposed near to an auricle and outside of an ear canal, and acquiring a core body temperature measurement using the operatively coupled temperature sensor.

In accordance with another aspect, a core body temperature measurement device comprises: a temperature sensor; a head- or neck-mountable mechanical frame or pad configured to operatively couple the temperature sensor with skin overlaying the carotid artery or a major arterial branch thereof; and a readout controller configured to acquire a temperature measurement using the temperature sensor and to output a core body temperature based on the acquired temperature measurement.

One advantage resides in providing an accurate non-invasive core body temperature measurement.

Another advantage resides in providing extended non-invasive core body temperature monitoring over a period of hours, days, or longer.

Another advantage resides in providing a head-mountable core body temperature measurement apparatus that is comfortable for the patient and does not impede the patient's vision.

Another advantage resides in providing a head-mountable core body temperature measurement apparatus that does not obscure or block the patient's face.

Another advantage resides in providing a head-mountable core body temperature measurement apparatus that includes a plurality of temperature sensors to identify a position for acquiring a most accurate core body temperature.

Still further advantages of the present invention will be appreciated to those of ordinary skill in the art upon reading and understand the following detailed description.

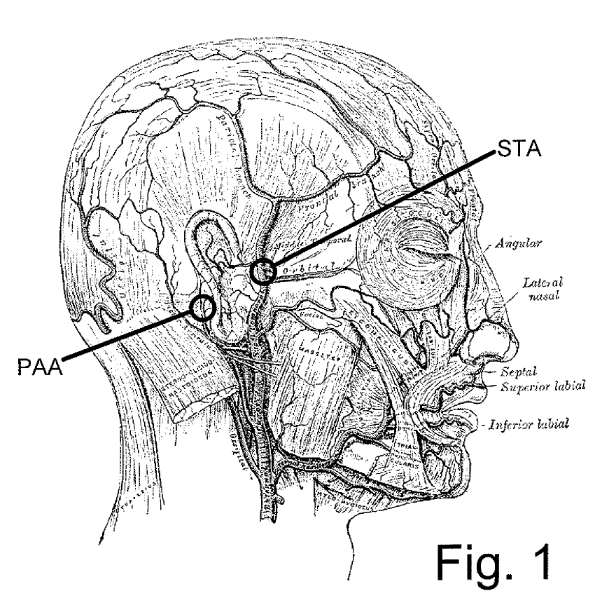

FIG. 1 diagrammatically shows a side view of a human head with the skin and other outer tissue removed to reveal arteries of the right side of the face and scalp, and further indicating preferred locations for acquiring non-invasive core body temperature measurements.

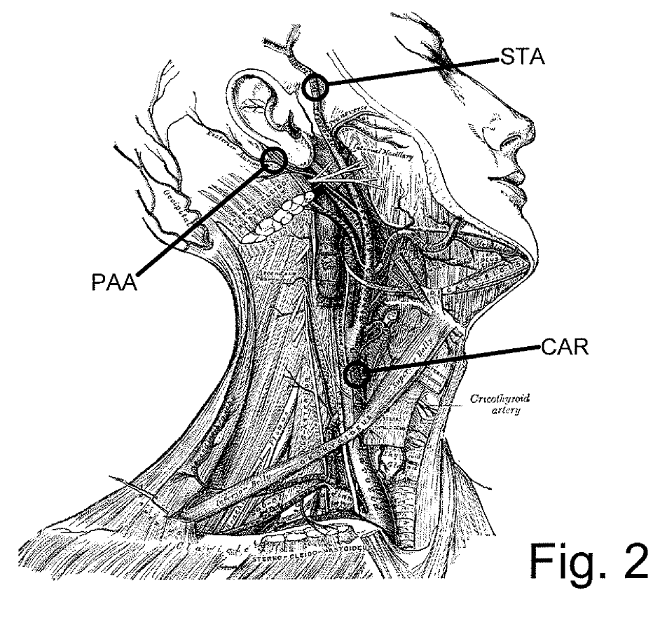

FIG. 2 diagrammatically shows a side view of a human neck supporting a partially turned human head, with the skin and other outer tissues partially removed to reveal arteries of the right side of the neck and head, and further indicating preferred locations for acquiring non-invasive core body temperature measurements.

FIG. 3 diagrammatically shows a readout controller for a core body temperature measurement device.

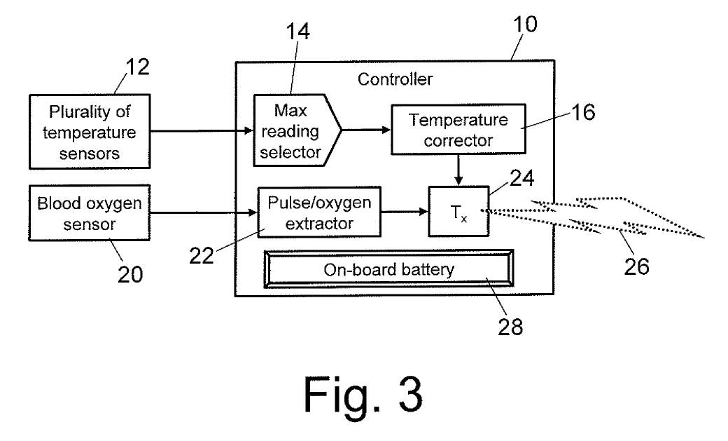

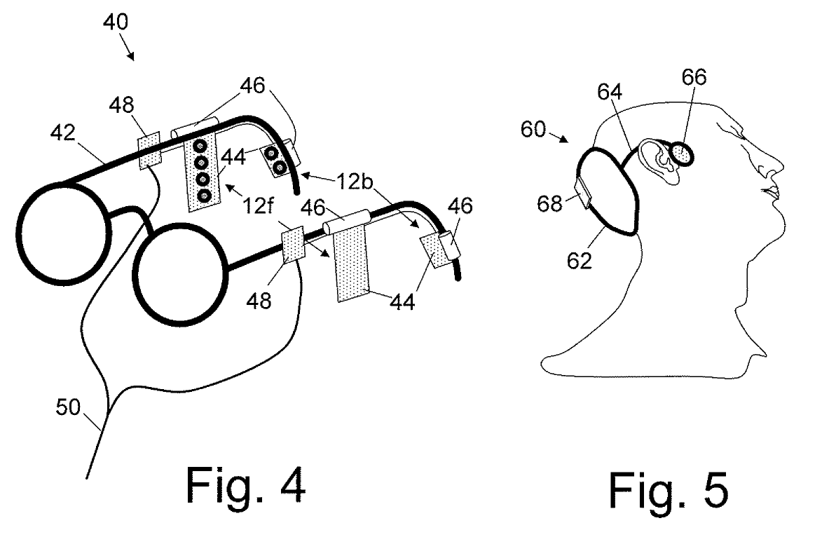

FIG. 4 diagrammatically shows a core body temperature measurement device including a mechanical frame in the form of an eyeglasses frame.

FIG. 5 diagrammatically shows a core body temperature measurement device including a mechanical frame in the form of a behind-the-head pillow having extensions configured to loop over the left and right auricles.

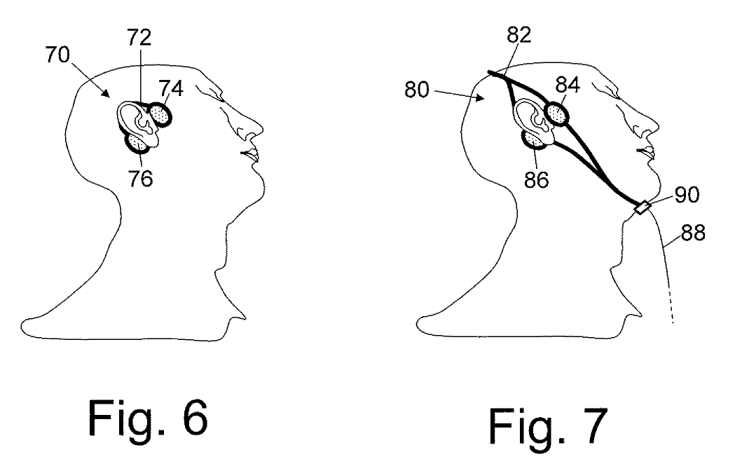

FIG. 6 diagrammatically shows a core body temperature measurement device including a mechanical frame in the form of headset including an earloop disposed around a proximate auricle without a headband.

FIG. 7 diagrammatically shows a core body temperature measurement device including a mechanical frame in the form of a circumferential headband.

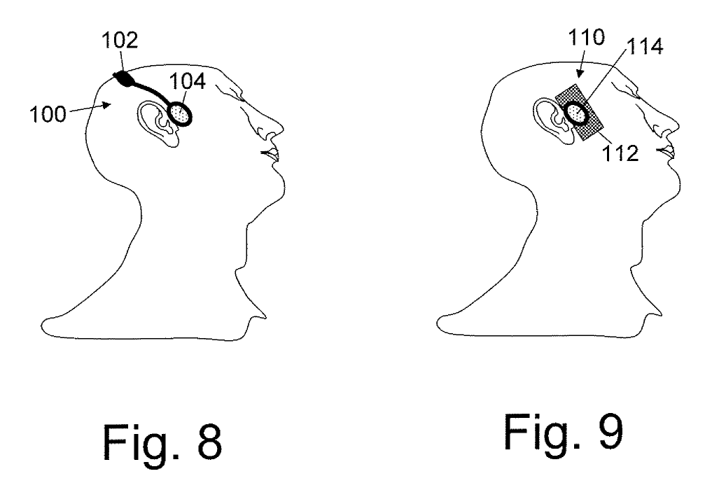

FIG. 8 diagrammatically shows a core body temperature measurement device including a mechanical frame in the form of a generally hemispherical headband.

FIG. 9 diagrammatically shows a core body temperature measurement device including a mechanical frame in the form of an adhesive pad.

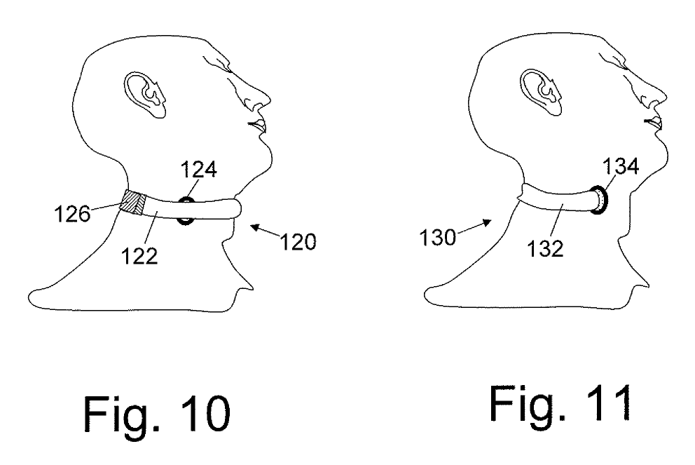

FIG. 10 diagrammatically shows a core body temperature measurement device including a mechanical frame in the form of an elastic neckband.

FIG. 11 diagrammatically shows a core body temperature measurement device including a mechanical frame in the form of a half-neckband.

With reference to FIGS. 1 and 2, the auricle, also known as the pinna, is the outer, projecting portion of the ear, that is, the visible part of the ear that resides outside of the head. It is recognized herein that arterial blood-rich superficial regions disposed near the auricle and outside the ear canal provide good sites for acquiring core body temperature measurements. For example, the superficial temporal artery is positioned forward of the auricle and carries arterial blood from the external carotid artery outward toward the surface of the scalp in front of the auricle. Accordingly, a temperature measurement device may be operatively coupled with skin overlaying a portion of a superficial temporal artery disposed anterior (that is, in front of) the auricle, such as at a region STA indicated in FIGS. 1 and 2. As another example, arterial vessels disposed behind the auricle, such as the posterior auricular artery, carry arterial blood from the external carotid artery outward toward the surface of the scalp behind the ear. Accordingly, a temperature measurement device may be operatively coupled with skin overlaying a portion of an artery ascending posterior to (that is, behind) the auricle, such as at the region PAA indicated in FIGS. 1 and 2.

While FIGS. 1 and 2 show the configuration of the aforementioned arteries, auricle, and other anatomical features for the right auricle, it is to be understood that bilateral symmetry pertains, and similar core body temperature measurement positions exist for the left auricle as well. Indeed, in some embodiments core body temperature measurements are acquired from regions disposed near both the left and right auricles.

With reference to FIG. 3, a suitable readout controller 10 for a core body temperature measurement device is described. The readout controller 10 reads temperature measurements using a temperature sensor or, in some embodiments, a plurality of temperature sensors 12, that are operatively coupled with skin overlaying an arterial blood-rich superficial region disposed near to an auricle and outside of an ear canal. For example, the temperature sensors 12 may be coupled with the region STA of FIGS. 1 and 2, the region PAA of FIGS. 1 and 2, or both, with the corresponding regions proximate to the left auricle, or with some combination thereof.

An advantage of providing the plurality of temperature sensors 12, rather than a single temperature sensor, is that the plurality of temperature sensors 12 can sample different portions of the skin. The precise location of the region STA, or of the region PAA, may vary slightly from person to person and may be difficult to pinpoint precisely on a given human head. In a suitable approach for addressing this problem, the plurality of sensors are read by the readout controller 10 and a maximum reading selector 14 selected the highest temperature measurement acquired by the plurality of temperature sensors 12 as the temperature reading for determining the core body temperature. This approach relies on the recognition made herein that the measured temperature should be highest at that point where the skin temperature most closely approximates the core body temperature. Lower temperature measurements generally reflect thermal losses due to lower thermal conductance of the skin in areas away from the skin overlaying an arterial blood-rich superficial region. Lower temperature measurements may also reflect inaccurate temperature readings due to poor contact of the temperature sensor with the skin or other measurement errors. Thus, by using the plurality of temperature sensors 12 and employing the maximum reading selector 14 to select the highest temperature measurement, such difficulties are alleviated. In some embodiments, the plurality of temperature sensors 12 are disposed proximate to both left and right auricles. In some embodiments, the plurality of temperature sensors 12 are disposed both anterior and posterior to an auricle. In some embodiments, the plurality of temperature sensors 12 are disposed both anterior and posterior to both left and right auricles. Although the approach using the plurality of temperature sensors 12 has advantages, it is also contemplated to employ a single temperature sensor to acquire a single temperature measurement, and to omit the maximum reading selector 14.

The acquired temperature measurement is expected to be close to the core body temperature due to the high level of superficial arterial blood flow just under the skin overlaying the arterial blood-rich superficial region disposed near the auricle. However, some difference between the acquired temperature measurement and the core body temperature can be expected due to thermal losses across the skin. Optionally, a temperature corrector 16 corrects the acquired temperature measurement for this temperature drop across the skin to generate the measured core body temperature. In some embodiments, the correction is an approximate correction based on an expected temperature drop across the skin. For example, it is typical to have about a 1° C. difference between the core body temperature and the skin temperature, due to thermal losses across the skin. Hence, in some embodiments the temperature corrector 16 adds 1° C. (or about 1.8° F.) to the skin temperature (e.g., the highest temperature measurement acquired by the plurality of temperature sensors 12 as selected by the maximum reading selector 14) to generate the core body temperature. For example, if the highest temperature measurement acquired by any of the temperature sensors 12 is 97.3° F., then the core body temperature is estimated as 97.3° F.+1.8° F.=99.1° F. As another example, if the highest temperature measurement is 37.1° C., then the core body temperature is estimated as 38.1° C. Optionally, the temperature corrector 16 performs other corrections or adjustments of the core temperature reading, such as a units conversion, for example, from a thermocouple voltage to degrees Celsius and/or degrees Fahrenheit, correction for non-linearity or other pre-determined systematic errors of the temperature sensors 12, or so forth.

With continuing reference to FIG. 3, optionally the measurement device includes sensors to acquire other physiological parameters. For example, a blood oxygen sensor 20, such as an SpO₂ sensor or an StO₂ sensor, acquires a measurement (typically an optically based measurement in the case of an SpO₂ or StO₂ sensor) that is converted into a blood oxygenation level reading and a pulse reading by a pulse/oxygen extractor 22. Different or additional sensors can be included, such as a blood pressure sensor.

The resulting information including the core body temperature and optional other readings such as blood oxygenation and pulse are output by a suitable output path such as a wired connection, an illustrated wireless transmitter 24 or transceiver that outputs a wireless data signal 26, or so forth. The core body temperature measurement device optionally includes other features. For example, if the core body temperature data is offloaded using a wired connection, then the wired connection can incorporate a power input lead to power the sensors 12, 20 and processors 14, 16, 22. Alternatively, if the illustrated wireless transmitter 24 or transceiver is used such that the core body temperature measurement device is a wireless device, then an on-board battery 28, power capacitor, or other on-board electrical power supply is suitably included.

As mentioned previously, the optional skin temperature corrector 16 in some embodiments employs an estimated skin temperature drop correction, such as a 1° C. temperature drop correction. This approach is computationally straightforward, but can lead to some error since the actual skin temperature drop varies based on factors such as moisture (e.g., sweat), ambient temperature, air convection, and so forth. To accommodate such factors, in some embodiments the skin temperature corrector 16 employs a more complex corrective approach based on feedback. For example, the one or more skin temperature sensors 12 can each include parallel conductive plates or films spaced apart by a distance that is adjustable using inchworm actuators, MEMS actuators, or so forth. By acquiring temperature measurements across the two plates at different plate separations, the heat flux can be determined from which the skin temperature drop can be determined. Designating the temperatures of the two conductive plates as T₁ and T₂, respectively, and the core body temperature as T_{core}, a system of equations is defined by:

$$\frac{d T}{d t} = α \frac{{d}^{2} T}{d {x}^{2}} , \qquad ( 1 )$$

where α=λ/ρcₚ, λ denotes thermal conductivity, ρ denotes density, and cₚ denotes specific heat. In a suitable coordinate system, x denotes depth with x=0 corresponding to a point inside the body at temperature T_{core} and x=hₛ corresponding to the surface of the skin. The boundary conditions for Equation (1) include the core body temperature T_{core} (to be determined) at x=0, and the measured temperature Tₛ at x=hₛ, that is, at the surface of the skin. If the plate at temperature T₂ is contacting the skin, then Tₛ=T₂ to a good approximation. The heat flux out of the skin is denoted qₛ herein.

Assuming the skin 104 can be represented as a plane of thickness hₛ and thermal conductivity λₛ, the heat flux out of the skin qₛ (that is, heat transfer rate on a per-unit area basis) can be written as:

$${q}_{s} = - λ \frac{d T}{d x} at x = {h}_{s} , \qquad ( 2 )$$

and a solution of Equation (1) can be approximated as:

$${T}_{core} = {T}_{s} + \frac{{h}_{s}}{{λ}_{s}} {q}_{s} + \frac{{h}_{s}^{2}}{2 {α}_{s}} \frac{d {T}_{s}}{d t} . \qquad ( 3 )$$

At equilibrium, Equation (3) reduces to:

$${T}_{core} = {T}_{s} + \frac{{h}_{s}}{{λ}_{s}} {q}_{s} , \qquad ( 4 )$$

which demonstrates that the core body temperature T_{core} is higher than the skin temperature by a temperature drop across the skin corresponding to (hₛ/λₛ)·qₛ.

By using feedback control of the actuators separating the parallel conductive plates or films, the values of the quantities Tₛ, qₛ, and

$$\frac{d {T}_{s}}{d t}$$

can be measured for different moments in time tᵢ={t₁, . . . , tₙ}ₜₒ produce a matrix of coupled equations:

$$[ \begin{matrix}1 & - {ξ}_{1} & - {η}_{1} \\ & … & \\ 1 & - {ξ}_{n} & - {η}_{n}\end{matrix} ] [ \begin{matrix}{T}_{core} \\ \frac{{h}_{s}}{{λ}_{s}} \\ \frac{{h}_{s}^{2}}{2 {α}_{s}}\end{matrix} ] = [ \begin{matrix}{T}_{s} ( {t}_{1} ) \\ ⋮ \\ {T}_{s} ( {t}_{n} )\end{matrix} ] , \qquad ( 5 )$$

in which the unknown quantities are T_{core},

$$\frac{{h}_{s}}{{λ}_{s}} and \frac{{h}_{s}^{2}}{2 {α}_{s}} ,$$

and where:

$$\begin{matrix}ξ ≡ {q}_{s} ( {t}_{i} ) , \\ and & ( 6 ) \\ η ≡ \frac{d {T}_{s}}{d t} ( {t}_{i} ) . & ( 7 )\end{matrix}$$

It is assumed here that T_{core},

$$\frac{{h}_{s}}{{λ}_{s}} and \frac{{h}_{s}^{2}}{2 {α}_{s}}$$

are time-independent during the time interval {t₁, . . . , tₙ} over which the set of measurements are acquired. The system of Equations (5) can be solved by the temperature corrector 16 using a least squares minimisation (LMS) procedure or other suitable coupled equations solver to provide the body core temperature T_{core}, and also the heat flux qₛ through the surface of the skin. The sampling moments tᵢ are suitably chosen such that to ensure that the system of Equations (5) is well-conditioned.

As yet another approach, the temperature corrector 16 can make a skin temperature drop correction determined based on physiological measurements such as the ambient temperature (suitably acquired using a temperature sensor that is not in contact with or close to the skin), skin sheet resistance or conductivity (measurable using a first electrode pair driving a small current and a second electrode pair measuring voltage generated by the drive current), or so forth. A lookup table or empirical formula suitably relates the skin temperature drop correction to the measured ambient temperature, skin sheet resistance, or other parameters.

In general, the core body temperature measurement device includes the one or more temperature sensors 12, the readout controller 10, and a head-mountable mechanical frame configured to operatively couple the temperature sensor or sensors 12 with skin overlaying an arterial blood rich superficial region disposed near to an auricle and outside of an ear canal. The readout controller 10 can either be mounted on the head-mountable mechanical frame, or can be disposed away from the frame and connected with the temperature sensors 12 via a wireless or wired link.

With reference to FIGS. 4-9, several head-mountable mechanical frames are set forth as illustrative examples.

FIG. 4 diagrammatically shows a core body temperature measurement device 40 including a mechanical frame in the form of an eyeglasses frame 42. The eyeglasses frame 42 can contain prescriptive lenses for correcting eyesight, or can contain non-corrective lenses, or can have no lenses at all. A first set of temperature sensors 12f are mounted near the left and right bends of the frame and are operatively coupled with skin overlaying portion of the superficial left and right temporal arteries anterior to the left and right auricles. A second set of temperature sensors 12b are mounted near the left and right earpieces and are operatively coupled with skin overlaying portions of left and right arteries ascending posterior to the left and right auricles. The temperature sensors 12f, 12b are mounted on supports 44 that each include a spring bias 46 coupling the support to the eyeglasses frame and pressing the supported temperature sensors against the skin overlaying the target arterial blood-rich superficial region.

The readout controller is suitably embodied by microchips 48 disposed on the eyeglasses frame 42 as illustrated. Wired connections 50 provide power to the microchips 48 and sensors 12f, 12b and provide a pathway for offloading the acquired core body temperature measurements and optional blood oxygenation or other measurements. An advantage of the wired connection 50 is that the core body temperature measurement device 40 does not need an on-board battery or other on-board power supply, which enables the core body temperature measurement device 40 to be lightweight. Although four sets of temperature sensors 12f, 12b are illustrated (a set of temperature sensors front and back of each auricle) it is contemplated to have fewer sets of temperature sensors. For example, the back temperature sensors 12b may be omitted, or temperature sensors may be coupled with skin overlaying an arterial blood rich superficial region on only the left side, or on only the right side. Moreover, the microchips 48 are optionally omitted and the readings of the temperature sensors 12f, 12b offloaded directly via the wired connection 50 to a readout processor that is not mounted on the eyeglasses frame 42.

FIG. 5 diagrammatically shows a core body temperature measurement device 60 including a mechanical frame in the form of a behind-the-head pillow 62 having extensions 64 configured to loop over the left and right auricles (only the right-side extension 64 is visible in FIG. 5). One or more temperature sensors are mounted on one or more supports 66 disposed on one or both extensions 64. In the illustrated embodiment, the supports 66 are positioned at ends of the extensions 64 located anterior to the left and right auricles, and couple the temperature sensors with skin overlaying portions of the left and right superficial temporal arteries. Additionally or alternatively, supports (not shown) can be arranged on the extensions 64 to couple temperature sensors with skin overlaying portions of arteries ascending posterior to an auricles. Optionally, a microchip 68 defining the readout controller 10 is disposed on or in the behind-the-head pillow 62 and operatively connects with the temperature sensors on the supports 66 via wires (not shown) running inside of or along the extensions 64.

FIG. 6 diagrammatically shows a core body temperature measurement device 70 including a mechanical frame in the form of headset including an earloop 72 disposed around a proximate auricle without a headband. The illustrated embodiment includes a first temperature sensor support 74 disposed anterior to the right auricle and coupling one or more temperature sensors with skin overlaying a portion of the right superficial temporal artery, and a second temperature sensor support 76 disposed posterior to the right auricle and coupling one or more temperature sensors with skin overlaying a portion of an artery ascending posterior to the right auricle. FIG. 6 shows the core body temperature measurement device 70 including the illustrated earloop 72 disposed around the right auricle; however, the earloop may be disposed around the left auricle instead, or an earloop may be disposed around each of the left and right auricles. The illustrated core body temperature measurement device 70 is a wireless device, and accordingly includes the readout controller 10 (FIG. 3) with the on-board battery 28 or other on-board power source and wireless transmitter 24 or transceiver mounted on the earloop 72. Some suitable on-board power devices and transmitters are known and used in existing wireless Bluetooth headsets that are sometimes embodied as earloops.

FIG. 7 diagrammatically shows a core body temperature measurement device 80 including a mechanical frame in the form of a circumferential headband 82 with one or more supports for one or more temperature sensors disposed on the circumferential headband proximate to one or both auricles and contacting skin overlaying one or more arterial blood rich superficial regions disposed near the proximate auricle or auricles. In the illustrated embodiment, a front support 84 is disposed anterior to the right auricle and couples one or more temperature sensors with skin overlaying a portion of the right superficial temporal artery, and a back temperature sensor support 86 is disposed posterior to the right auricle and couples one or more temperature sensors with skin overlaying a portion of an artery ascending posterior to the right auricle. Optionally, corresponding supports for temperature sensors are also provided proximate to the left auricle. In some embodiments, only one of the two supports 84, 86 are included. The illustrated core body temperature measurement device 80 includes a wired connection 88 for offloading core body temperature measurements and optionally other measurements, and for supplying electrical power to the device 80. An illustrated readout controller 90 is disposed under the chin on the circumferential headband 80 where it is readily connected with the wired connection 88, and the readout controller 90 connects with the temperature sensors on the supports 84, 86 via wires running through or along portions of the circumferential headband 82. In other embodiments, the readout controller can be disposed elsewhere on the headband 82, such as at the top of the head, or can be disposed away from the circumferential headband 82 and connected with the temperature sensors via the wired connection 88.

FIG. 8 diagrammatically shows a core body temperature measurement device 100 including a mechanical frame in the form of a generally hemispherical headband 102 having an end with a temperature sensor support 104 disposed anterior to the right auricle and coupling one or more temperature sensors with skin overlaying a portion of the right superficial temporal artery. Although not shown, a second or alternative support may be disposed behind the right auricle to couple one or more temperature sensors with skin overlaying a portion of an artery ascending posterior to the right auricle. Also not shown in the right-profile view of FIG. 8 is an optional corresponding temperature sensor support or supports proximate to the left auricle. The readout controller is suitably mounted on top of the hemispherical head 102 (not shown in the perspective view of FIG. 8) and optionally includes the wireless transmitter 24 or transceiver.

FIG. 9 diagrammatically shows a core body temperature measurement device 110 including a mechanical frame in the form of an adhesive pad 112 adhered to contact skin overlaying a portion of the right superficial temporal artery. One or more temperature sensors are suitably disposed on, under, or in the adhesive pad 112 in thermal communication with the skin. In the illustrated embodiment, a rigid disk 114 contains the one or more temperature sensors along with a readout controller suitably conforming with the readout controller 10 of FIG. 3. Optionally, an additional or alternative adhesive pad may be disposed behind the right auricle to couple one or more temperature sensors with skin overlaying a portion of an artery ascending posterior to the right auricle. Also not shown in the right-profile view of FIG. 9 is an optional corresponding one or more adhesive pads coupling one or more temperature sensors proximate to the left auricle.

The mechanical frames illustrated in FIGS. 4-9 are examples. Other head-mounted mechanical frames may be used that are configured to operatively couple one or more temperature sensors with skin overlaying an arterial blood-rich superficial region disposed near to an auricle and outside of an ear canal. Moreover, it is to be appreciated that the core body temperature measurement methods disclosed herein may be practiced in other ways besides through the use of a head-mountable mechanical frame. For example, a hand-held temperature sensor 12 may be held by a physician, nurse, or other person and manually coupled with a patient's skin STA, PAA overlaying an arterial blood rich superficial region disposed near to an auricle and outside of an ear canal, and the core body temperature measurement acquired using the operatively coupled temperature sensor.

With returning reference to FIG. 2, skin overlaying an arterial blood-rich superficial region STA, PAA disposed near to an auricle and outside of an ear canal is identified herein as an effective place to measure core body temperature, due to the close proximity of flowing arterial blood at a temperature near the core body temperature. For example, the region STA overlays a portion of a superficial temporal artery, while the region PAA overlays a portion of an artery ascending posterior to an auricle. Both these arteries are major branches of the carotid artery. More generally, the temperature sensor may be coupled with skin overlaying the carotid artery or a major arterial branch thereof. For example, at a region CAR on the neck the carotid artery is relatively near to the surface. The region CAR is well-known as a suitable location for acquiring a pulse measurement, for example by pressing the fingers onto the region CAR to feel the pulse flowing through the carotid artery. A high volume of blood flows through the carotid artery in the neck, and this blood is flowing from the heart and accordingly is at a temperature near the core body temperature. Accordingly, it is contemplated herein to place the one or more temperature sensors 12 at the region CAR or elsewhere along the carotid artery or its major arterial branches that are close to the surface.

With reference to FIGS. 10 and 11, two illustrative neck-mountable mechanical frames for positioning the temperature sensors are illustrated. FIG. 11 shows a core body temperature measurement device 120 including a mechanical frame in the form of an elastic neckband 122 that goes around the neck to support a temperature sensor support 124 at the region CAR on the right side of the neck. Although not visible in FIG. 10, such a temperature sensor support can also be disposed on the left side of the neck. The illustrated elastic neckband 122 includes a Velco® fastener 126 or other fastener to enable the neckband 122 to be strapped snugly around the neck to moderately press the temperature sensor support 124 against the region CAR. The fastener 126 is advantageously located at the back of the neck for patient comfort. Although not shown, the core body temperature measurement device 120 suitably includes a controller such as the controller 10 of FIG. 3, for example embedded in or externally mounted on the neckband 122.

FIG. 11 shows another illustrative core body temperature measurement device 130 including a mechanical frame in the form of an elastic half-neckband 132 that goes around the back of the neck and terminates at left and right ends (only the right end being illustrated) at the region CAR to support a temperature sensor support 134 at the region CAR on the right (and/or optionally left) side of the neck. The half-neckband 132 preferably has a semi-rigid form or built-in spring (not shown) to bias and retain the half-neckband 132 snugly around the neck to moderately press the temperature sensor support 134 against the region CAR. Although not shown, the core body temperature measurement device 130 suitably includes a controller such as the controller 10 of FIG. 3, for example embedded in or externally mounted on the half-neckband 132.

The neck-mountable mechanical frames illustrated in FIGS. 10 and 11 are examples, and other neck-mountable mechanical frames may be used that are configured to operatively couple one or more temperature sensors with skin overlaying a portion of the carotid artery in the neck. For example, in other embodiments a pad similar to the adhesive pad 112 of FIG. 9 is attached at the region CAR to couple one or more temperature sensors to the region CAR.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A core body temperature measurement device comprising:
a temperature sensor;
a head-mountable mechanical frame or pad configured to operatively couple the temperature sensor with skin overlaying an arterial blood-rich superficial region disposed near to an auricle and outside of an ear canal; and
a readout controller configured to acquire a temperature measurement using the temperature sensor and to output a core body temperature based on the acquired temperature measurement.

2. The core body temperature measurement device as set forth in claim 1, wherein the temperature sensor comprises a plurality of temperature sensors each configured to operatively couple with a different portion of skin overlaying an arterial blood-rich superficial region disposed near to the auricle and outside of the ear canal, and the readout controller is configured to acquire temperature measurements using the plurality of temperature sensors and to output a core body temperature based on the temperature measurements.

3. The core body temperature measurement device as set forth in claim 1, wherein the head-mountable mechanical frame comprises:
an eyeglasses frame having left and right bends generally passing over respective left and right auricles and each terminating in an earpiece, the temperature sensor being disposed at or near at least one of:
the left bend and operatively coupling with skin overlaying a portion of a superficial temporal artery anterior to the left auricle;
the right bend and operatively coupling with skin (STA) overlaying a portion of a superficial temporal artery anterior to the right auricle;
the left earpiece and operatively coupling with skin overlaying a portion of an artery ascending posterior to the left auricle, and
the right earpiece and operatively coupling with skin overlaying a portion of an artery ascending posterior to the right auricle.

4. The core body temperature measurement device as set forth in claim 3, wherein the head-mountable mechanical frame further comprises:
a support for the temperature sensor; and
a spring bias coupling the support to the eyeglasses frame, the spring bias pressing the temperature sensor against skin overlaying an arterial blood-rich superficial region disposed near to the auricle and outside of the ear canal.

5. The core body temperature measurement device as set forth in claim 1, wherein the head-mountable mechanical frame comprises:
a behind-the-head pillow having extensions configured to loop over left and right auricles, the temperature sensor being disposed on one or both extensions in thermal communication with skin overlaying an arterial blood-rich superficial region disposed near to the auricle around which the extension loops.

6. The core body temperature measurement device as set forth in claim 5, wherein the readout controller is disposed on or in the pillow.

7. The core body temperature measurement device as set forth in claim 1, wherein the head-mountable mechanical frame comprises:
a headset that includes an earloop disposed around a proximate auricle without a headband, the temperature sensor being disposed on the earloop in thermal communication with skin overlaying an arterial blood-rich superficial region disposed near to the proximate auricle around which the earloop is disposed.

8. The core body temperature measurement device as set forth in claim 1, wherein the head-mountable mechanical frame comprises:
a circumferential headband, the temperature sensor being disposed on the circumferential headband proximate to an auricle and in thermal communication with skin overlaying an arterial blood-rich superficial region disposed near the proximate auricle.

9. The core body temperature measurement device as set forth in claim 1, wherein the head-mountable mechanical frame comprises:
a generally hemispherical headband having an end disposed proximate to an auricle, the temperature sensor being disposed on the end proximate to the auricle and in thermal communication with skin overlaying an arterial blood-rich superficial region disposed near to the proximate auricle.

10. The core body temperature measurement device as set forth in claim 1, wherein the head-mountable mechanical frame or pad comprises:
an adhesive pad adhered to skin overlaying an arterial blood-rich superficial region disposed near a proximate auricle, the temperature sensor being disposed on, under, or in the adhesive pad in thermal communication with said skin.

11. The core body temperature measurement device as set forth in claim 1, further comprising:
a blood oxygen sensor, the head-mountable mechanical frame or pad further configured to operatively couple the blood oxygen sensor with skin overlaying an arterial blood-rich superficial region disposed near to an auricle and outside of an ear canal, the readout controller further configured to acquire a blood oxygen measurement using the blood oxygen sensor.

12. The core body temperature measurement device as set forth in claim 1, wherein the temperature sensor comprises:
a temperature sensor operatively coupling with skin overlaying a portion of a superficial temporal artery.

13. The core body temperature measurement device as set forth in claim 1, wherein the temperature sensor comprises:
a temperature sensor operatively coupling with skin overlaying a portion of an artery ascending posterior to an auricle.

14. The core body temperature measurement device as set forth in claim 1, wherein the temperature sensor comprises:
a front temperature sensor operatively coupling with skin overlaying a portion of a superficial temporal artery disposed anterior of a selected auricle; and
a rear temperature sensor operatively coupling with skin overlaying a portion of an artery ascending posterior to the selected auricle.

15. The core body temperature measurement device as set forth in claim 1, wherein the readout controller comprises:
a temperature corrector configured to correct the temperature measurement acquired using the temperature sensor for at least a temperature drop across the skin to generate the core body temperature.

16. The core body temperature measurement device as set forth in claim 1, wherein the readout controller is disposed on or in the head-mountable mechanical frame or pad.

17. The core body temperature measurement device as set forth in claim 16, further comprising:
an electrical power source disposed on or in the head-mountable mechanical frame or pad; and
a wireless transmitter or transceiver disposed on or in the head-mountable mechanical frame or pad and configured to wirelessly output the core body temperature off the mechanical frame or pad.

18. A core body temperature measurement method comprising:
operatively coupling a temperature sensor with skin overlaying an arterial blood-rich superficial region disposed near to an auricle and outside of an ear canal; and
acquiring a core body temperature measurement using the operatively coupled temperature sensor.

19. The core body temperature measurement method as set forth in claim 18, wherein the operative coupling includes:
operatively coupling a temperature sensor with skin overlaying a portion of a superficial temporal artery.

20. The core body temperature measurement method as set forth in claim 18, wherein the operative coupling includes:
operatively coupling a temperature sensor with skin overlaying a portion of an artery ascending posterior to an auricle.

21. The core body temperature measurement method as set forth in claim 18, wherein the operative coupling includes operatively coupling a plurality of temperature sensors with skin overlaying an arterial blood-rich superficial region disposed near an auricle, and the acquiring of the core body temperature measurement includes:
acquiring a plurality of temperature measurements using the plurality of spaced-apart temperature sensors; and
deriving the core body temperature measurement from the highest temperature measurement of the acquired plurality of temperature measurements.

22. The core body temperature measurement method as set forth in claim 18, wherein the acquiring of the core body temperature measurement includes:
acquiring a temperature measurement using the operatively coupled temperature sensor; and
adjusting the acquired temperature measurement to account for thermal losses in the skin to generate the core body temperature measurement.

23. The core body temperature measurement method as set forth in claim 18, wherein the operatively coupling of a temperature sensor with skin overlaying an arterial blood-rich superficial region disposed near to an auricle and outside of an ear canal includes:
disposing the temperature sensor on a head-mountable mechanical frame; and
disposing the head-mountable mechanical frame on a head.

24. A core body temperature measurement device comprising:
a temperature sensor;
a head- or neck-mountable mechanical frame or pad configured to operatively couple the temperature sensor with skin overlaying the carotid artery or a major arterial branch thereof; and
a readout controller configured to acquire a temperature measurement using the temperature sensor and to output a core body temperature based on the acquired temperature measurement.

25. The core body temperature measurement device as set forth in claim 24, wherein the temperature sensor comprises a plurality of temperature sensors each configured to operatively couple with a different portion of the skin overlaying the carotid artery or a major arterial branch thereof, and the readout controller is configured to acquire temperature measurements using the plurality of temperature sensors and to output a core body temperature based on the temperature measurements.

26. The core body temperature measurement device as set forth in claim 24, wherein the skin overlays an arterial blood-rich superficial region disposed near to an auricle and outside of an ear canal, and the head- or neck-mountable mechanical frame is a head-mountable frame selected from a group consisting of:
an eyeglasses frame having left and right bends generally passing over respective left and right auricles and each terminating in an earpiece, the temperature sensor being disposed at or near at least one of a bend and an earpiece,
a behind-the-head pillow having extensions configured to loop over left and right auricles, the temperature sensor being disposed on one or both extensions in thermal communication with skin overlaying an arterial blood-rich superficial region disposed near to the auricle around which the extension loops,
a headset that includes an earloop disposed around a proximate auricle without a headband, the temperature sensor being disposed on the earloop in thermal communication with skin overlaying an arterial blood-rich superficial region disposed near to the proximate auricle around which the earloop is disposed,
a circumferential headband, the temperature sensor being disposed on the circumferential headband proximate to an auricle and in thermal communication with skin overlaying an arterial blood-rich superficial region disposed near the proximate auricle, and
a generally hemispherical headband having an end disposed proximate to an auricle, the temperature sensor being disposed on the end proximate to the auricle and in thermal communication with skin overlaying an arterial blood-rich superficial region disposed near to the proximate auricle.

27. The core body temperature measurement device as set forth in claim 24, wherein the skin overlays a region (CAR) of the neck through which a portion of the carotid artery passes, and the head- or neck-mountable mechanical frame is an elastic neckband configured to strap around the neck, the temperature sensor being disposed with the elastic neckband and pressed by the elastic neckband against the region of the neck through which a portion of the carotid artery passes.

28. The core body temperature measurement device as set forth in claim 24, wherein the skin overlays a region of the neck through which a portion of the carotid artery passes, and the head- or neck-mountable mechanical frame is a half-neckband configured to wrap around a backside of the neck, the temperature sensor being disposed at one or both ends of the half-neckband and pressed by the half-neckband against the region of the neck through which a portion of the carotid artery passes.

29. The core body temperature measurement device as set forth in claim 24, wherein the skin overlays a region of the neck through which a portion of the carotid artery passes, and the head- or neck-mountable mechanical frame is a neck-mountable frame that positions the temperature sensor against the region through which a portion of the carotid artery passes.

30. The core body temperature measurement device as set forth in claim 24, wherein the readout controller comprises:
a temperature corrector configured to correct the temperature measurement acquired using the temperature sensor for at least a temperature drop across the skin to generate the core body temperature.
